(19) United States
(12) Patent Application Publication
(10) Pub. No.: US 2023/0414862 A1
(43) Pub. Date: Dec. 28, 2023

(54) INLINE PUMP SYSTEM

(71) Applicant: ClearPoint Neuro, Inc., Solana Beach, CA (US)

(72) Inventors: Jeremy Stigall, Solana Beach, CA (US); Nathan Williams, Solana Beach, CA (US); Kamal Vij, Solana Beach, CA (US)

(21) Appl. No.: 18/342,038

(22) Filed: Jun. 27, 2023

Related U.S. Application Data
(60) Provisional application No. 63/355,916, filed on Jun. 27, 2022.

(51) Int. Cl. A61M 5/142; A61M 5/168

(52) U.S. Cl. CPC A61M 5/14216; A61M 5/16881; A61M 2210/0693

(57) ABSTRACT

A therapeutic delivery system includes a cannula configured to deliver a therapeutic into an anatomical region, the cannula having an internal lumen and a pumping system coupled to the cannula. The pumping system is inline of a therapeutic flow pathway between a therapeutic source and the cannula. The pumping system is configured to drive the therapeutic from the therapeutic source and toward the cannula.

### CROSS-REFERENCE TO RELATED APPLICATION

The present application claims priority to U.S. Provisional Patent Application Ser. No. 63/355,916, filed Jun. 27, 2022, entitled “INLINE PUMP SYSTEM,” the full disclosure of which is incorporated herein by reference.

### BACKGROUND

There is a need for pumping systems that are configured for use in an MRI conditional scenario including for use in delivering small, precise volumes of therapeutics into an anatomical region (such as a brain).

### SUMMARY

Disclosed is an inline pumping apparatus that connects to a cannula to deliver therapeutic directly to a patient such as in an MRI conditional environment.

In one aspect, a therapeutic delivery system includes a cannula configured to deliver a therapeutic into an anatomical region, the cannula having an internal lumen and a pumping system coupled to the cannula. The pumping system is inline of a therapeutic flow pathway between a therapeutic source and the cannula. The pumping system is configured to drive the therapeutic from the therapeutic source and toward the cannula.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic representation of a neurointervention system that utilizes an inline pump apparatus.

FIG. 2 shows a schematic representation of an example embodiment of an inline pump apparatus.

FIGS. 3 and 4 show alternate embodiment of the inline pump apparatus.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed is an inline pumping apparatus that connects to a cannula to deliver therapeutic directly to a patient such as in an MRI conditional environment.

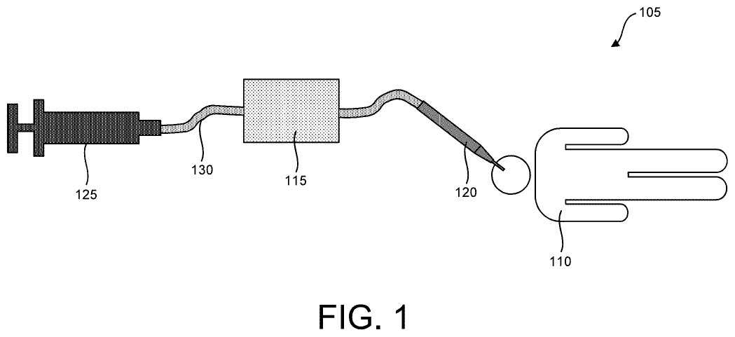

FIG. 1 shows a schematic representation of an inline therapeutic delivery system 105 that is configured to infuse or otherwise deliver therapeutic to a patient 110, such as a human patient. The system 105 includes a therapeutic metering or pumping system 115 that is positioned inline of a therapeutic delivery pathway that includes a cannula 120 that interfaces with an anatomical region of a patient. In a non-limiting example, the cannula 120 can be deployed to a brain of a patient such as via a transcranial pathway. However, it should be appreciated that the cannula can be configured to interact with and deliver therapeutic to any anatomical region of a patient.

The cannula 120 is fluidly coupled to a therapeutic reservoir 125 (i.e., a therapeutic source such as a container) via a tubing 130. The cannula 120 infuses therapeutic from the reservoir 125 (via the tubing) into the brain of the patient. As mentioned, the pumping system 115 is positioned inline of the tubing 130. The pumping system 115 is configured to deliver the therapeutic at a prescribed volume and flowrate each of which can be controlled by a user. The pumping system 115 is fluidly coupled (such as by being attached to or in close proximity) to the cannula 120. Although described herein in the context of the system intervening with and delivering a therapeutic to the brain, it should be appreciated that the system can intervene with other parts of the anatomy and is not limited to use with the brain. For example, the system can be used to interact with cerebrospinal fluid. The tubing can interposed between the pumping system and the cannula along the fluid flow pathway.

In an embodiment, the cannula 120 is an elongated body having an internal lumen that communicates with a therapeutic reservoir 125. The cannula has a distal end region that can be positioned in the brain for delivery of therapeutic into the brain via an opening at the distal region of the cannula 120. The cannula 120 can vary in configuration and can be, for example, a needle, catheter or any other device that is used such that a lumen is positioned within the patient to deliver therapeutic to a desired location via the cannula 120.

The therapeutic reservoir 125 can vary in configuration and can be any type of reservoir or container configured to contain or store a therapeutic or any substance to be delivered to the brain. In non-limiting example embodiments, the therapeutic reservoir 125 is a syringe, capsule or any other device that holds therapeutic. In another embodiment, a therapeutic can be administered directly into the pumping system via an inlet in the pumping system.

The configuration of the tubing 130 can also vary. The tubing 130 can be any device having a lumen or passageway positioned between the therapeutic reservoir 125 and the cannula 120 to transport therapeutic therebetween.

The pumping system 115 is configured to passively allow controlled amounts of fluid or force controlled amounts of fluid to flow through the pumping system 115 toward the cannula 120. Figuring 2 shows a non-limiting, example embodiment of a pumping system 115. The pumping system 115 includes a housing 205 that defines a central fluid fill chamber 210 contained within the housing. A piston 215 is movably positioned within the fill chamber 210. Input tubing 220 communicates with the fill chamber 210 via an inlet such that the therapeutic reservoir 125 is in fluid communication with the fill chamber via the inlet and the input tubing 220. An input valve 225 is coupled to the inlet and regulates fluid flow from the input tubing 220 into the fill chamber 210 such that the input valve 225 permits fluid flow into the fill chamber 210 and blocks fluid flow out of the fill chamber 210 via the input tubing 220. A spring can be couped to the input valve such as to bias the input valve into a closed state that blocks fluid flow out of the fill chamber.

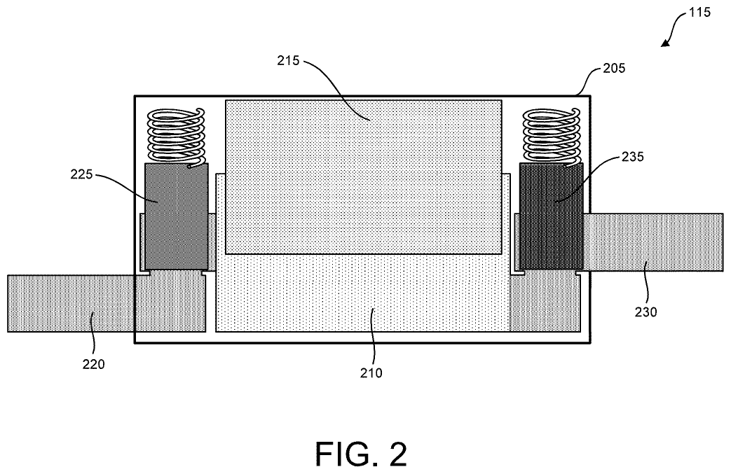

With reference still to FIG. 2, an output tubing 230 communicates with the fill chamber 210 via an outlet. An output valve 235 is coupled to the outlet and regulates fluid flow out of the fill chamber 210 such that the output valve 235 permits fluid flow out of the fill chamber 210 and blocks fluid flow into the fill chamber 210 via the output tubing 230. The outlet provides fluid communication between the fill chamber 210 and the cannula 120 via the output tubing 230. A spring can be couped to the output valve such as to bias the output valve into a closed state that blocks fluid flow into the fill chamber. The output valve can open to permit flow out of the fil chamber upon a pressure threshold being reached in the fill chamber to thereby permit fluid flow out of the fill chamber via the outlet.

In use, the piston 215 moves through the fill chamber 210 (such as in an up and down or side to side manner) such that a hollow volume of the fill chamber 210 can be increased or decreased based on a position of the piston 215. The piston 215 causes a pressure differential that draws fluid from the input tubing (which is connected to the therapeutic reservoir 125) into the fill chamber 210. The piston can also move to cause a pressure differential that forces fluid out of the fill chamber. The input valve 225 and the output valve 235 regulate fluid flow such that that fluid always flows from the input tubing 220 into the fill the chamber 210 and always flows to the output tubing 230 when exiting the fill chamber 210.

The materials used in system may be selected such that it can be used in an MRI environment. The system may be configured for single use or can be reusable. The pumping system 115 can be incorporated into the cannula such as sharing a common structure. For example, the housing 205 of the pumping system 115 can be integrated or monolithically formed with at least a portion of the cannula. Or the pumping system can be a separate module that is removable or fixedly attached to the cannula.

The movement in the pumping system may be controlled by standard motors, solenoids, PZT, MEMS, capacitive mechanisms, mechanical (clockwork) or any other mechanism. Moreover, the mechanisms may deliver quick ‘bolus’ doses or may be slowed to provide more uniform and constant dosing. Parallel mechanisms may be employed with offset timing to provide even more consistent dosing. In addition, the valves may be passive valve (e.g. driven by springs for example) or active valves (e.g. opened/closed via controlled mechanisms).

In addition, although the above embodiment shows the fluid being driven by a chamber that is opened/closed (such that the empty volume is increased/decreased) other mechanisms can be used to drive fluid. Such other mechanisms include for example gear, lobe, vane, roller, screw, peristaltic, and diaphragm mechanisms.

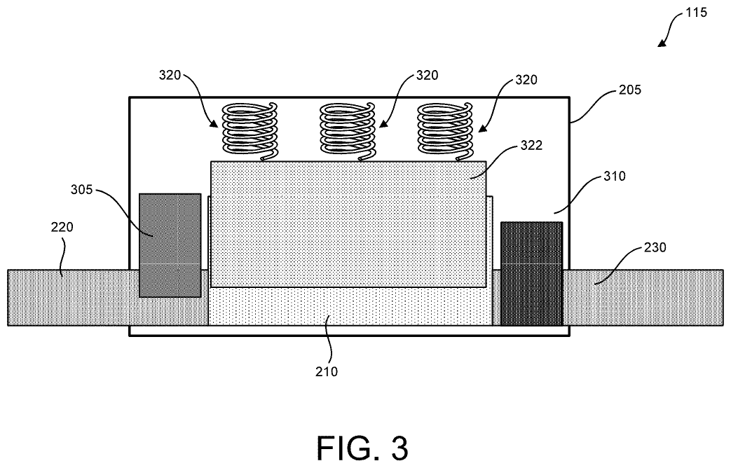

FIG. 3 shows an alternative embodiment of the pumping system 115. This embodiment includes a controlled input valve 305 coupled to the input tubing 220 and a controlled output valve 310 coupled to the output tubing 230. The valves control fluid entry and exit of a ‘passive’ fill chamber 210. The incoming therapeutic is pressurized (such as via a pressured gas or a spring loaded syringe plunger) prior to entering the pumping system 115. When the input valve 305 is open it allows therapeutic from the input tubing 220 to enter the fill chamber 210. When the input valve 305 is closed and the output valve 310 is opened then the therapeutic exits through the output tubing 230. The system can be configured such that the output valve opens when the input valve closes and the input valve opens when the output valve closes. The system 115 of FIG. 3 includes a spring-loaded mechanism 320 configured to exert a force onto fluid in the fill chamber 210 via a plunger 322 to drain the fill chamber 210 when the output valve 310 is open. Alternative mechanisms (such as elastic membranes) are within the scope of this disclosure. The spring loaded mechanism 320 may be replaced with or supplemented with other mechanisms for exerting force onto the fluid in the fill chamber. For example, such other mechanisms can include pressurized fluid (such as a gas) or any other mechanism.

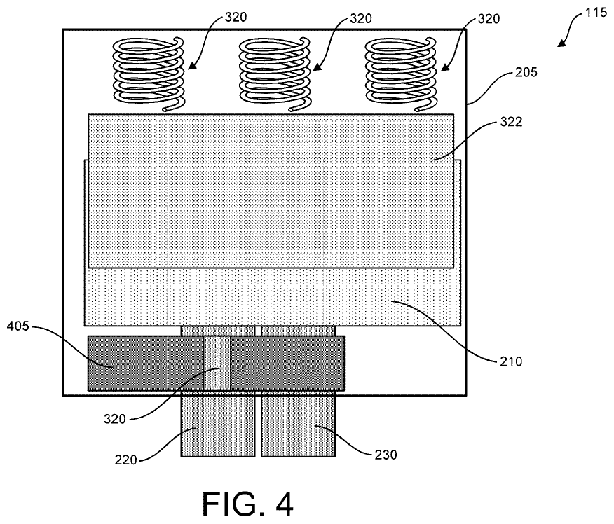

The embodiment of FIG. 3 can be further simplified so only one movement or valve is required. FIG. 4 shows an embodiment of a pumping system 115 wherein a sliding valve 405 has a single lumen 410. In this embodiment, the pumping system only has a single valve that controls fluid flow into and out of the fill chamber. The valve 405 is configured to move relative to the fill chamber 210. If the valve 405 is moved to the left then the fill chamber 210 is open to the input tubing 220 via the lumen 410 of the valve 405. If the valve 405 is moved to the right then the fill chamber 210 is open to the output tubing 230 via the lumen 410. Multiple lumens and different input/output spacing may also be utilized to accomplish the same filling/emptying with a single sliding valve.

Thus, the single valve is movably coupled to the inlet and the outlet, wherein the single valve has an opening through which fluid can flow from the input tubing into the fill chamber when the valve is in a first position. The valve blocks fluid flow out of the fill chamber via the output tubing when the valve is in the first position. The valve transitions to a second position (such as by sliding) wherein the fluid can flow from the fill chamber into the output tubing via the opening, and wherein the single valve blocks fluid flow into the fill chamber via the outlet tubing when the single valve is in the second position.

An alternative mechanism can incorporate a nozzle design that utilizes known fluid dynamic properties (fluid viscosity, pressure, etc.) such that the therapeutic travels through the nozzle at a known flow rate. A single valve may then be opened or closed to deliver the therapeutic in boluses at controlled times.

The described embodiments show components and chambers such that they are rigid structures. Flexible and dynamic structures may also be used (e.g. membranes).

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A therapeutic delivery system, comprising:
a cannula configured to deliver a therapeutic into an anatomical region, the cannula having an internal lumen;
a pumping system coupled to the cannula, the pumping system being inline of a therapeutic flow pathway between a therapeutic source and the cannula, the pumping system configured to drive the therapeutic from the therapeutic source and toward the cannula.

2. The system of claim 1, further comprising a tubing that fluidly connects the pumping system to the cannula such that the tubing is interposed between the pumping system and the cannula.

3. The system of claim 1, further comprising a tubing that fluidly connects the therapeutic source to the cannula such that the tubing is interposed between the therapeutic source and the cannula.

4. The system of claim 1, further comprising the therapeutic source.

5. The system of claim 4, wherein the therapeutic source is any device that holds a therapeutic.

6. The system of claim 1, wherein the pumping system is directly attached to the cannula.

7. The system of claim 1, wherein the pumping system and cannula are MRI compatible.

8. The system of claim 1, wherein the pumping system comprises:
an outer housing defining a fill chamber;
an inlet to the fill chamber, wherein the inlet provides fluid communication between the therapeutic source and the fill chamber via an input tubing;
an outlet from the fill chamber, wherein the outlet provides fluid communication between the fill chamber and the cannula via an output tubing;
an input valve coupled to the inlet, wherein the input valve regulates flow from the input tubing into the fill chamber such that the input valve permits fluid flow into the fill chamber and blocks fluid flow out of the fill chamber via the input tubing;
an output valve coupled to the outlet, wherein the output valve regulates flow from the fill chamber into the output tubing such that the output valve permits fluid flow out of the fill chamber and blocks fluid flow into the fill chamber via the outlet tubing;
a mechanism coupled to the fill chamber, wherein the mechanism causes a pressure differential that draws fluid via the inlet into the fill chamber or pushes fluid out of the fill chamber via the outlet.

9. The system of claim 8, wherein the mechanism is a piston movably positioned within the fill chamber, wherein the piston moves through the fill chamber to cause the pressure differential.

10. The system of claim 8, wherein the mechanism is a gear, lobe, vane, roller, screw, peristaltic, or diaphragm.

11. The system of claim 8, wherein the pumping system and the cannula share a common structure.

12. The system of claim 8, wherein the therapeutic is pressurized prior to entering the fill chamber.

13. The system of claim 1, wherein the pumping system comprises:
an outer housing defining a fill chamber;
an inlet to the fill chamber, wherein the inlet provides fluid communication between the therapeutic source and the fill chamber via an input tubing;
an outlet from the fill chamber, wherein the outlet provides fluid communication between the fill chamber and the cannula via an output tubing;
a single valve movably coupled to the inlet and the outlet, wherein the single valve has one or more openings through which fluid can flow from the input tubing into the fill chamber when the valve is in a first position, and wherein the valve blocks fluid flow out of the fill chamber via the output tubing when the valve is in the first position;
and wherein the single valve transitions to a second position wherein the fluid can flow from the fill chamber into the output tubing via the opening, and wherein the single valve blocks fluid flow into the fill chamber via the outlet tubing when the single valve is in the second position;
a mechanism coupled to the fill chamber, wherein the mechanism causes a pressure differential that draws fluid via the inlet into the fill chamber or pushes fluid out of the fill chamber via the outlet.

14. The system of claim 13, wherein the single valve slides between the first position and the second position.

15. The system of claim 14, wherein the pumping system and the cannula share a common structure.

16. The system of claim 1, wherein the anatomical region is a brain and wherein the therapeutic is delivered along a transcranial pathway.

17. The system of claim 14, wherein the mechanism pushes fluid into the fill chamber.

18. The system of claim 14, wherein the mechanism causes fluid to be sucked into the fill chamber.

19. The system of claim 11, wherein the outer housing of the pumping system shares a common structure with the cannula.
